(19) United States
(12) Patent Application Publication
(10) Pub. No.: US 2015/0056311 A1
(43) Pub. Date: Feb. 26, 2015

(54) SORGHUM EXTRACT AND ITS THERAPEUTIC USES

(71) Applicants: Brian Pellens, Bakersfield, CA (US); D. Edward Settle, Fort Collins, CO (US)

(72) Inventors: Brian Pellens, Bakersfield, CA (US); D. Edward Settle, Fort Collins, CO (US)

(21) Appl. No.: 14/463,390

(22) Filed: Aug. 19, 2014

Related U.S. Application Data
(60) Provisional application No. 61/868,009, filed on Aug. 20, 2013.

(51) Int. Cl. A61K 36/899; A23L 1/30; A23L 2/52

(52) U.S. Cl. CPC A61K 36/899; A23L 2/52; A23L 1/3002; A23V 2002/00
USPC 424/750

(57) ABSTRACT

The present disclosure relates to Sorghum extract and its uses. The present disclosure also provides methods for making the Sorghum extract.

Free radicals such as reactive oxygen species and reactive nitrogen species are a natural consequence of metabolism of organisms and can be increased by exposure to radiation and carcinogens. This is thought to increase the risk of numerous diseases including inflammatory disorders, proliferative disorders, and the like. Therefore, there is a need for therapies that can scavenge free-radicals. One such therapy comprises antioxidants, a class of compounds that can suppress the oxidation of other molecules.

Many bioactive compounds such as phenolic acids, flavonoids and stillbenoids are antioxidants which can be grouped largely into polyphenolics. Polyphenols are widely distributed in the plant kingdom. Sweet Sorghum is an agricultural crop grown mainly for sugar syrup production. Grain Sorghums are grown mainly for the starch content of their seeds. Biomass Sorghums are grown mainly for the plant mass. Each of these Sorghums is of the species Sorghum bicolor. Sorghums comprise polyphenols. However, there is little information available to the content of the total phenolics and antioxidant activity in different parts of Sorghum. Thus a need exists for antioxidants that have high polyphenol content. The present disclosure shows some parts of Sorghum have higher polyphenolics than the other parts. Targeted extraction of specific parts of Sorghum may produce cost effective and high polyphenolic content extracts that exhibits better therapeutic effects.

Sorghum also comprises vitamins, minerals, and dietary fiber. A diet enriched with dietary fiber is helpful in digestion and absorption thereby providing glycemic control. Benefits of a diet comprising additional vitamins and minerals are well known in the literature. Therefore, a need exists to provide additional vitamins, minerals, and dietary fiber to supplement a regular diet.

The present disclosure relates to a Sorghum bicolor (L.) Moench extract, comprising total phenolics of value, as determined by the Folin-Ciocalteu assay, at least greater than about 0.1 mg Gallic Acid Equivalent per gram Fresh Weight (GAE/g FW), and that has an antioxidant activity, as determined by the Trolox Equivalent Antioxidant Capacity (TEAC) method using ABTS (2,2′-azino-bis(3-ethylbenzthiazoline-6-sulfonic acid) as an colorimetric indicator compound, of at least greater than about 0.01 mg Trolox Equivalent per gram Fresh Weight (TE/g FW).

In an embodiment, Sorghum bicolor (L.) Moench extract is obtained by extracting Sorghum bicolor (L.) Moench with C1-C7 organic solvents, for example n-heptane.

In an embodiment, the Sorghum bicolor (L.) Moench extract is obtained by extracting Sorghum bicolor (L.) Moench with C1-C6 polar solvents.

In yet another embodiment, the Sorghum bicolor (L.) Moench extract is obtained by extracting Sorghum bicolor (L.) Moench with aqueous C1-C6 polar solvents.

In another embodiment, the Sorghum bicolor (L.) Moench extract is obtained by extracting Sorghum bicolor (L.) Moench with C1-C6 polar solvents such as alcohol. In certain aspect, the alcohol is ethanol.

In yet another embodiment, Sorghum bicolor (L.) Moench extract is obtained by extracting Sorghum bicolor (L.) Moench with aqueous C1-C6 polar solvents. In a further aspect, the aqueous C1-C6 polar solvents comprise from about 1% to about 40% water and the rest is C1-C6 polar solvent. In certain aspect, the aqueous polar solvent comprises about 20% water and the rest is polar solvent.

In an embodiment, the disclosure provides a Sorghum extract wherein the total phenolics value, as determined by the Folin-Ciocalteu assay, is at least greater than about 4.0 mg GAE/g FW and the antioxidant activity, as determined by the ABTS assay, is greater than about 0.4 mg TE/g FW.

In yet another embodiment, the disclosure provides a Sorghum extract according wherein the total phenolics value, as determined by the Folin-Ciocalteu assay, is at least greater than about 3.5 mg GAE/g FW and the antioxidant activity, as determined by the ABTS assay, is greater than about 0.4 mg TE/g FW.

In another embodiment, the disclosure provides a Sorghum extract, wherein the total phenolics value, as determined by the Folin-Ciocalteu assay, is at least greater than about 0.9 mg GAE/g FW and antioxidant activity, as determined by the ABTS assay, of at least greater than about 0.5 mg TE/g FW.

The present disclosure also relates to a process for making a Sorghum extract comprising the steps of:
a. contacting a member selected from the group consisting of Sorghum whole plant, Sorghum grain, Sorghum seed head, Sorghum leaves, Sorghum pith, Sorghum juice, Sorghum dermal layer, Sorghum rind (with or without dermal layer removed) and mixtures thereof with a solvent at a temperature below 180 degrees Fahrenheit such that certain compounds which are measurable under the Folin-Ciocalteu or TEAC assay methods are mass transferred from the member to the solvent; and
b. separating insoluble solids from the solvent obtained in step (a).

In an embodiment, the disclosure provides a process for making a Sorghum extract further comprising the step of grinding of the said members before or after contacting said members with the solvent.

In an embodiment, the disclosure provides a process for making a Sorghum extract further comprising the steps:
c. selectively removing the solvent through methods such as settling, filtration, evaporation and the like; and
d. obtaining the Sorghum extract.

In an embodiment, the disclosure provides a process for making a Sorghum extract further comprising the steps:
c. selectively removing the solvent through methods such as settling, filtration, evaporation and the like; and
d. further purifying the product of step (a) through methods such as chromatography, adsorption, and the like; and
e. obtaining the Sorghum extract.

In certain embodiments, the disclosure provides a process for making a Sorghum extract wherein the solvent is selected from the group consisting of alcohol, supercritical CO₂, acetone, water and hexane or a mixture thereof.

In an embodiment, the disclosure provides a process for making a Sorghum extract wherein the solvent is aqueous ethanol.

In an embodiment, the disclosure provides a process for making a Sorghum extract wherein the solvent is about 60% aqueous ethanol to about 99% aqueous ethanol.

In an embodiment, the disclosure provides a process for making a Sorghum extract wherein the solvent is about 80% aqueous ethanol.

In an embodiment, the disclosure provides a process for making a Sorghum extract further comprising the step of purifying the Sorghum extract to obtain a purified Sorghum extract.

In an embodiment, the disclosure provides a process for making a Sorghum extract, wherein the Sorghum extract comprises total phenolics further wherein the total phenolics as determined by the Folin-Ciocalteu assay have a value at least greater than about 0.1 mg GAE/g FW.

In an embodiment, the disclosure provides a process for making a Sorghum extract, wherein the Sorghum extract exhibits an antioxidant activity as determined by the ABTS assay of at least greater than about 0.01 mg TE/g FW

In an embodiment, the disclosure provides a process for making a Sorghum extract wherein the member is Sorghum leaf.

In an embodiment, the disclosure provides a process for making a Sorghum extract, wherein the member is Sorghum leaf and the Sorghum extract comprises total phenolics further wherein the total phenolics as determined by the Folin-Ciocalteu assay have a value at least greater than about 4.0 mg GAE/g FW.

In an embodiment, the disclosure provides a process for making a Sorghum extract, wherein the member is Sorghum leaf and the Sorghum extract exhibit an antioxidant activity as determined by the ABTS assay of at least greater than about 0.4 mg TE/g FW.

In an embodiment, the disclosure provides a process for making a Sorghum extract, wherein the member is Sorghum seed head.

In an embodiment, the disclosure provides a process for making a Sorghum extract wherein the Sorghum seed head comprises total phenolics further wherein the total phenolics as determined by the Folin-Ciocalteu assay have a value at least greater than about 3.5 mg GAE/g FW.

In an embodiment, the disclosure provides a process for making a Sorghum extract wherein the Sorghum seed head exhibit an antioxidant activity as determined by the ABTS assay of at least greater than about 0.4 mg TE/g FW.

In an embodiment, the disclosure provides a process for making a Sorghum extract wherein the member is Sorghum dermal layer.

In an embodiment, the disclosure provides a process for making a Sorghum extract, wherein the member is Sorghum dermal layer and the Sorghum extract comprises total phenolics further wherein the total phenolics as determined by the Folin-Ciocalteu assay have a value at least greater than about 0.9 mg GAE/g FW.

In an embodiment, the disclosure provides a process for making a Sorghum extract, wherein the member is Sorghum dermal layer and the Sorghum extract exhibit an antioxidant activity as determined by the ABTS assay of at least greater than about 0.5 mg TE/g FW.

The present disclosure provides a food comprising Sorghum bicolor (L.) Moench extract, comprising total phenolics of a value, as determined by the Folin-Ciocalteu assay, of at least greater than about 0.1 mg Gallic Acid Equivalent per gram Fresh Weight (GAE/g FW), and that has an antioxidant activity, as determined by the Trolox Equivalent Antioxidant Capacity (TEAC) method using ABTS (2,2′-azino-bis(3-ethylbenzthiazoline-6-sulfonic acid) as an colorimetric indicator compound, of at least greater than about 0.01 mg TE/g FW.

The present disclosure provides a beverage comprising Sorghum bicolor (L.) Moench extract, comprising total phenolics of a value, as determined by the Folin-Ciocalteu assay, at least greater than about 0.1 mg Gallic Acid Equivalent per gram Fresh Weight (GAE/g FW), and that has an antioxidant activity, as determined by the Trolox Equivalent Antioxidant Capacity (TEAC) method using ABTS (2,2′-azino-bis(3-ethylbenzthiazoline-6-sulfonic acid) as an colorimetric indicator compound, of at least greater than about 0.01 mg (TE/g FW).

The present disclosure provides a nutraceutical comprising Sorghum bicolor (L.) Moench extract, comprising total phenolics of value, as determined by the Folin-Ciocalteu assay, at least greater than about 0.1 mg Gallic Acid Equivalent per gram Fresh Weight (GAE/g FW), and that has an antioxidant activity, as determined by the Trolox Equivalent Antioxidant Capacity (TEAC) method using ABTS (2,2′-azino-bis(3-ethylbenzthiazoline-6-sulfonic acid) as an colorimetric indicator compound, of at least greater than about 0.01 mg (TE/g FW).

The present disclosure provides a composition comprising Sorghum bicolor (L.) Moench extract, comprising total phenolics of value, as determined by the Folin-Ciocalteu assay, at least greater than about 0.1 mg Gallic Acid Equivalent per gram Fresh Weight (GAE/g FW), and that has an antioxidant activity, as determined by the Trolox Equivalent Antioxidant Capacity (TEAC) method using ABTS (2,2′-azino-bis(3-ethylbenzthiazoline-6-sulfonic acid) as an colorimetric indicator compound, of at least greater than about 0.01 mg (TE/g FW).

The Sorghum extract of the present disclosure is also useful as prophylactic or therapeutic agents for treating diseases or disorders related to proliferative diseases and inflammatory diseases.

Accordingly, another aspect of the invention provides methods of treating or preventing diseases or conditions described herein by administering to a mammal, such as a human, a therapeutically effective amount of Sorghum extract as described herein in an amount effective to treat or prevent said disorder or disease.

The present disclosure provides a method of administering Sorghum extract.

The phrase “therapeutically effective amount” means an amount of extract of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. The amount of Sorghum extract that will correspond to such an amount will vary depending upon factors such as the disease condition and its severity, the identity (e.g., weight) of the mammal in need of treatment, the concentration or dilution of the extract, the method of delivery, but can nevertheless be routinely determined by one skilled in the art.

The terms “treat” and “treatment” refer to both therapeutic treatment and prophylactic or preventative measures, wherein the object is to prevent or slow down (lessen) an undesired physiological change or disorder.

As used herein, the term “aqueous ethanol” refers to ethanol comprising water. For example, 70% aqueous ethanol refers to ethanol comprising 30% water by volume. Thus a 100 ml 70% aqueous ethanol comprises 30% water or 30 ml water.

As used herein, the term “mammal” refers to a warm-blooded animal that has or is at risk of developing a disease described herein and includes, but is not limited to, guinea pigs, dogs, cats, rats, mice, hamsters, and primates, including humans.

As used herein, the term “Sorghum extract” refers to Sorghum bicolor (L.) Moench extract or “extract.”

As used here the term “Sorghum ” refers to Sorghum bicolor (L.) Moench.

The Sorghum extract of the present disclosure may be administered by any convenient route, e.g. into the gastrointestinal tract (e.g. rectally or orally), the nose, lungs, musculature or vasculature or transdermally. The Sorghum extract of the present disclosure may be administered in any convenient administrative form, e.g. tablets, powders, capsules, solutions, dispersions, suspensions, syrups, sprays, suppositories, gels, emulsions, patches, etc. The compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the disclosure.

The references to certain embodiments made in the following description are considered illustrative only of the principles of the disclosure. Further, since numerous modifications and changes will readily be apparent to those skilled in the art, it is not intended to limit the disclosure to the exact construction and process shown as described herein. Accordingly, all suitable modifications and equivalents may be resorted to as falling within the scope of the disclosure and as defined by the claims that follow.

The words “comprise,” “comprising,” “include,” and “including” when used in this specification and in the following claims are intended to specify the presence of the stated features, integers, components, or steps, but they do not preclude the presence or addition of one or more additional features, integers, components, or steps thereof.

General terms used in any of the embodiments herein can be defined as follows; however, the meaning stated should not be interpreted as limiting the scope of the term per se.

### BRIEF DESCRIPTION OF THE DRAWINGS

FIG. 1 shows results for total phenolics of the pith and dermal layer for both Dale and M81EM81E varieties. Results for total phenolics are displayed as milligram gallic acid equivalents per gram of fresh weight material extracted (mg GAE/g FW) with standard error bars. Percentages of phenolics and antioxidant activity based on source (pith & dermal) are included above the bars.

FIG. 2 shows results for antioxidant activity of the pith and dermal layer for both Dale and M81E varieties. Results for antioxidant activity are displayed as milligram Trolox equivalents per gram of fresh weight with standard error bars. Percentages of phenolics and antioxidant activity based on source (pith & dermal) are included above the bars.

FIG. 3 shows data for percent moisture analysis for pith and dermal layer of Dale and M81E varieties. Data is displayed as % weight by moisture with standard error bars

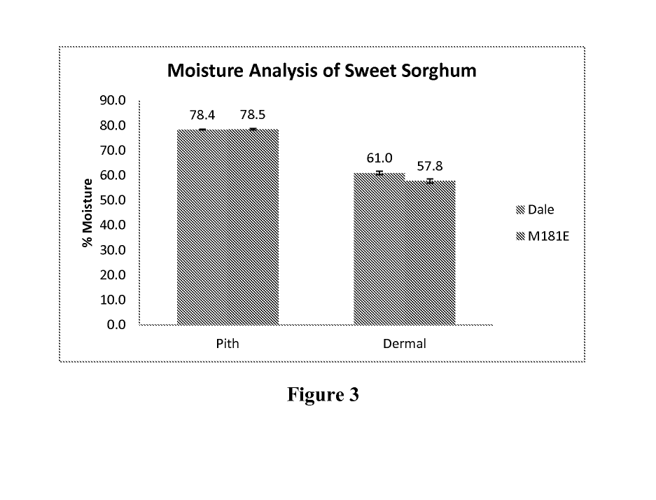

FIG. 4 shows total phenolic data for pith and dermal layer of Dale variety on a fresh weight basis and dry weight basis.

FIG. 5 shows total phenolic data for pith and dermal layer of M81E variety on a fresh weight basis and dry weight basis.

FIG. 6 shows Principle Component Analysis extracts based on their LC-MS profiles. The farther apart two points are, the more “different” they are based on principle components.

FIG. 7 shows the results from the proliferation assays obtained from cell count. Cell count was measured by a Nexlecom cellometer and cell count data is presented as % of solvent control. A cell count lower than the solvent control indicates suppression of cellular proliferation by the treatment. Solvent, solvent (80% ethanol) as a negative control: 5FU, 5-fluoro uracil as a positive drug control. Numbers next to treatment indicates the concentration of phenolic rich extract dosed in the assay in microgram gallic acid equivalents per milliliter of media in cell culture (μg GAE/ml). Values are presented as means±standard error (SEM).

FIG. 8 shows the results from the proliferation assays obtained from WST assay. The WST assay, light absorbance at 450 nm was measured and results presented as % of solvent control. An absorbance lower than the solvent control indicates suppression of cellular proliferation by the treatment. Solvent, solvent (80% ethanol) as a negative control: 5FU, 5-fluoro uracil as a positive drug control. Numbers next to treatment indicates the concentration of phenolic rich extract dosed in the assay in microgram gallic acid equivalents per milliliter of media in cell culture (μg GAE/ml). Values are presented as means±standard error.

FIG. 9 shows the data from the BrdU assay for HCT 116 cells. The lower the value, the more proliferation is being inhibited by sorghum extracts. Media, solvent (80% ethanol) as a negative control: 5FU, 5-fluoro uracil as a positive drug control. Number next to the treatment indicates the concentration (μg GAE/ml media) at which the human colon cancer cells were dosed. Values are presented as means±standard error. Arrows demonstrate a dose-dependency.

FIG. 10 demonstrates the proapoptotic activity of Dale dermal Sorghum extracts on HCT116 dosed for 24 hours. Proapoptotic activity was measured by Caspase Glo 3/7 assay for active caspase 3/7 activity. Lumens values for each treatment were measured and data are presented as % of solvent control. The higher the value, the more apoptosis is being induced by the treatment. Solvent, solvent (80% ethanol) as a negative control: 5FU, 5-fluoro uracil as a positive drug control. Numbers next to treatment indicates the concentration of phenolic rich extract dosed in the assay in microgram gallic acid equivalents per milliliter of media in cell culture (μg GAE/ml). Values are presented as means±standard error.

FIG. 11 displays data for total phenolics from Dale variety as determined by the Folin-Ciocalteu assay. Results expressed as means (n=3)±SEM in milligram gallic acid equivalents per gram of fresh weight material extracted (mg GAE/g FW).

FIG. 12 displays data for antioxidant activity from Dale variety as determined by the ABTS assay. Results are expressed as means (n=3)±SEM in milligram Trolox equivalents per gram of fresh weight (mg TE/g FW).

FIG. 13 shows dose response curves for HCT116 cells. Cells were treated for 24 hours with pith and dermal phenolic rich extracts prepared by extraction with either acetone (AE) or ethanol (EE). Response is based on cell proliferation as estimated by the cell counting. Simple linear regression was used to estimate IC₅₀ values. Values are presented as percent of solvent control (y axis) at a specific dose of the respective phenolic rich extract in μg GAE/mL (x axis).

FIG. 14 shows results when colon cancer stem cells were treated for 24 hours with Sorghum Dale variety phenolic extracts before being tested for proliferation by BrdU assay. Solvent, solvent control; 5FU, 5-fluorouracil; GSE+R, grape seed extract and resveratrol. Values for treatments are presented as means (n=3)±standard error.

FIG. 15 shows results when colon cancer stem cells were treated for 24 hours with Sorghum Dale variety phenolic extracts (35, 17.5, & 8.75 μg GAE/ml) before being counted by cellometer. 5FU, 5-fluorouracil; GSE+R, grape seed extract and resveratrol. Values for treatments are presented as means (n=2)±standard deviation.

FIG. 16 shows results when colon cancer stem cells were treated for 24 hours with Sorghum Dale variety phenolic extracts (35, 17.5, & 8.75 μg GAE/ml) before being tested for proliferation by Caspase 3/7 glo assay. 5FU, 5-fluorouracil; GSE+R, grape seed extract and resveratrol. Values for treatments are presented as means (n=2)±standard deviation.

FIG. 17 shows results when colon cancer stem cells were treated for 24 hours with sweet Sorghum Dale variety phenolic extracts (35, 17.5, & 8.75 μg GAE/ml) before being tested for apoptosis by TUNEL assay. 5FU, 5-fluorouracil; GSE+R, grape seed extract and resveratrol. Values for treatments are presented as means (n=2)±standard deviation.

FIG. 18 shows results when colon cancer stem cells were treated for 24 hours with sweet Sorghum Dale variety phenolic extracts (35, 17.5, & 8.75 μg GAE/ml) before being tested for colony forming ability by the Colony forming assay. 5FU, 5-fluoro uracil; GSE+R, grape seed extract and resveratrol. Values for treatments are presented as means (n=3)±standard error.

FIG. 19 shows results when colon cancer stem cells p53 shRNA were treated for 24 hours with sweet Sorghum Dale variety phenolic extracts (35, 17.5, & 8.75 μg GAE/ml) before being tested for proliferation by BrdU assay. 5FU, 5-fluorouracil; GSE 35+R, grape seed extract and resveratrol. Values for treatments are presented as means (n=3)±standard error.

FIG. 20 shows results when colon cancer stem cells p53 shRNA were treated for 24 hours with sweet Sorghum Dale variety phenolic extracts 35, 17.5, & 8.75 μg GAE/ml) before being counted by cellometer. 5FU, 5-fluorouracil; GSE+R, grape seed extract and resveratrol. Values for treatments are presented as means (n=2)±standard deviation.

FIG. 21 shows results when colon cancer stem cells p53 shRNA were treated for 24 hours with sweet Sorghum Dale variety phenolic extracts (35, 17.5, & 8.75 μg GAE/ml) before being tested for proliferation by Caspase 3/7 glo assay. 5FU, 5-fluorouracil; GSE+R, grape seed extract (35 μg/ml) and resveratrol (27 μM); D 35 dermal extract (35 μg GAE/ml). Values for treatments are presented as means (n=2)±standard deviation.

FIG. 22 shows results when colon cancer stem cells with p53 shRNA were treated for 24 hours with sweet Sorghum Dale variety phenolic extracts (35, 17.5, & 8.75 μg GAE/ml) before being tested for apoptosis by TUNEL assay. 5FU, 5-fluorouracil; GSE+R, grape seed extract and resveratrol. Values for treatments are presented as means±standard deviation.

FIG. 23 shows results when colon cancer stem cells with p53 shRNA were treated for 24 hours with sweet Sorghum Dale variety phenolic extracts (35, 17.5, & 8.75 μg GAE/ml) before being tested for colony forming ability by the Colony forming assay. 5FU, 5-fluorouracil; GSE+R, grape seed extract and resveratrol. Values for treatments are presented as means (n=3)±standard error.

FIG. 24 Total phenolics of sweet Sorghum components: Displays data for total phenolics for pith (comfith), dermal (dermax), leaf, seed head, and whole plant as determined by the Folin-Ciocalteu assay. Results expressed as means (n=3)±standard error bars in milligram gallic acid equivalents per gram of fresh weight material extracted (mg GAE/g FW).

FIG. 25 Antioxidant activity of sweet Sorghum components: Displays data for antioxidant activity of pith (comfith), dermal (dermax), leaf, seed head, and whole plant as determined by the ABTS assay. Results are expressed as means (n=3)±standard error bars in milligram Trolox equivalents per gram of fresh weight (mg TE/g FW).

FIG. 26 Anti-proliferative effects of sweet Sorghum components by BrdU Colon Cancer Stem cells were treated for 24 hours with sweet Sorghum extracts (35 μg GAE/ml) before being tested for proliferation by BrdU assay. Values for treatments are presented as means (n=3)±standard error.

FIG. 27 Pro-apoptotic effects of sweet Sorghum components by caspase. Colon cancer stem cells were treated for 24 hours with sweet Sorghum extracts (35 μg GAE/ml) before being tested for apoptosis by Caspase 3/7 glo assay. Values for treatments are presented as means (n=3)±standard error.

FIG. 28 Pro-apoptotic effects of sweet Sorghum components by TUNEL. Colon cancer stem cells were treated for 24 hours with sweet Sorghum extracts (35 μg GAE/ml) before being tested for apoptosis by TUNEL assay. Values for treatments are presented as means (n=3)±standard error.

FIG. 29 shows Total phenolic (TP in red) and antioxidant activity (AOA in blue) for variety 1 at different harvest times (X axis).

FIG. 30 shows Total phenolic (TP in red) and antioxidant activity (AOA in blue) for variety 2 at different harvest times (X axis).

FIG. 31 shows Total phenolic (TP in red) and antioxidant activity (AOA in blue) for variety 3 at different harvest times (X axis).

FIG. 32 shows Total phenolic (TP in red) and antioxidant activity (AOA in blue) for variety 4 at different harvest times (X axis).

FIG. 33 shows Total phenolic (TP in red) and antioxidant activity (AOA in blue) for variety 5 at different harvest times (X axis).

FIG. 34 shows average antioxidant activities for each of the variety 1-5 across 8 harvest times are presented with standard error.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

Embodiments disclosed herein are illustrated in greater detail by means of the non-limiting examples described below.

### EXAMPLES

The following examples are provided for illustrative purposes and only demonstrate certain aspects of the disclosure. These examples are not intended to limit the scope of the disclosure.

### Example 1

Analytical investigation of Dale and M81E varieties.

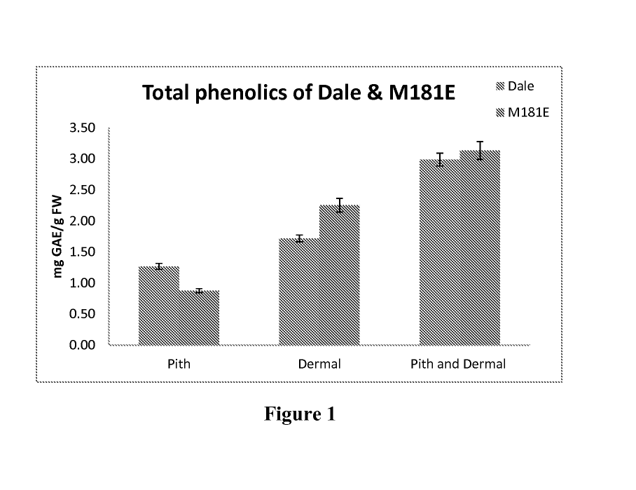

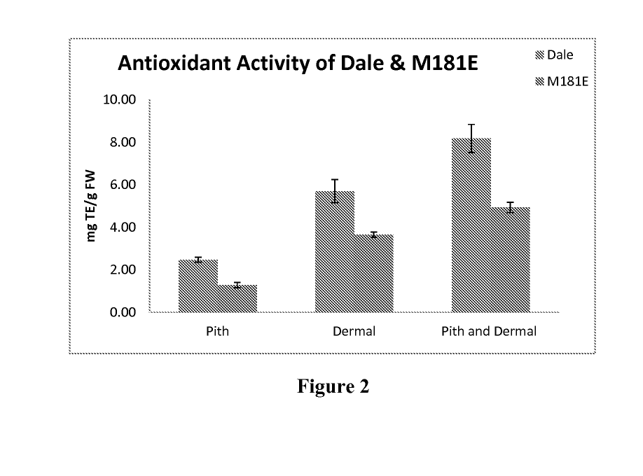

Methodology: Dale and M81E sweet Sorghum varieties were grown in Kern County, CA and processed into pith and dermal layer. Pith and dermal layers from both varieties were extracted thrice in 80% acetone after homogenization with an IKA T25 Digital Ultra Turrax Homogenizer. Extracts from the pith and dermal layers of both varieties were subjected to assays to quantify phenolic and antioxidant activity. Folin-Cioacalteu assay-quantification of phenolic constituents and ABTS assay-antioxidant activity. See FIG. 1 and FIG. 2 and Tables 1 and 2 for results.

Table 1: Displays results collected from Folin-Ciocalteu (FC) and ABTS assays. Results for the FC assay are reported as mean milligram gallic acid equivalents/gram of fresh weight±standard error (mg GAE/gfw±SE). Result for ABTS is displayed as mean milligram Trolox equivalents per gram fresh weight±standard error (mg TE/gfw±SE). NA=No Activity. *data collected from research from 1. Bröhan, M., Jerkovic, V. & Collin, S. Potentiality of red Sorghum for producing stilbenoid-enriched beers with high antioxidant activity. Journal of agricultural and food chemistry 59, 4088-94 (2011). **data collected from research by 2. Njongmeta, N. Extractability profiling and antioxidant activity of flavonoids in Sorghum grain and non-grain materials. East (2010) at <http://gradworks.umi.com/33/70/3370773.html>, refers to in-house comparisons from extracting fruits with similar protocols to understand how sweet Sorghum extracts compare to well-known phenolic/antioxidant rich foods.

TABLE 1

| Sample | FC | ABTS |
|---|---|---|
| Pith (Dale) | 1.27 ± 0.05 | 2.48 ± 0.12 |
| Dermal (Dale) | 1.72 ± 0.06 | 5.70 ± 0.54 |
| Pith (M81E) | 0.88 ± 0.03 | 1.28 ± 0.12 |
| Dermal (M81E) | 2.25 ± 0.11 | 3.65 ± 0.12 |
| Strawberries (in-house) | 1.13 ± 0.03 | 4.13 ± 0.06 |
| Blueberries (in-house) | 3.23 ± 0.04 | 5.42 ± 0.08 |
| Raspberries (in-house) | 1.70 ± 0.03 | 3.81 ± 0.06 |
| Blueberries (Literature) | 4.12* | 4.76 ± 0.29** |
| Cranberries (Literature) | 3.15* | 1.19 ± 0.08** |

Table 2: Displays results collected from Folin-Ciocalteu (FC), and ABTS assays on a dry weight basis. Results for the FC assay are reported as mean milligram gallic acid equivalents/gram of dry weight±standard error (mg GAE/gdw±SE). Results for ABTS assay is displayed as mean milligram Trolox equivalents per gram dry weight±standard error (mg TE/gdw±SE).

TABLE 2

| Sample | FC | ABTS |
|---|---|---|
| Pith (Dale) | 5.87 ± 0.22 | 11.44 ± 0.54 |
| Dermal (Dale) | 4.41 ± 0.14 | 14.61 ± 1.39 |
| Pith (M81E) | 4.09 ± 0.15 | 16.99 ± 0.57 |
| Dermal (M81E) | 5.34 ± 0.26 | 11.69 ± 0.28 |

### Example 2

Moisture Analysis of Pith and Dermal Layer

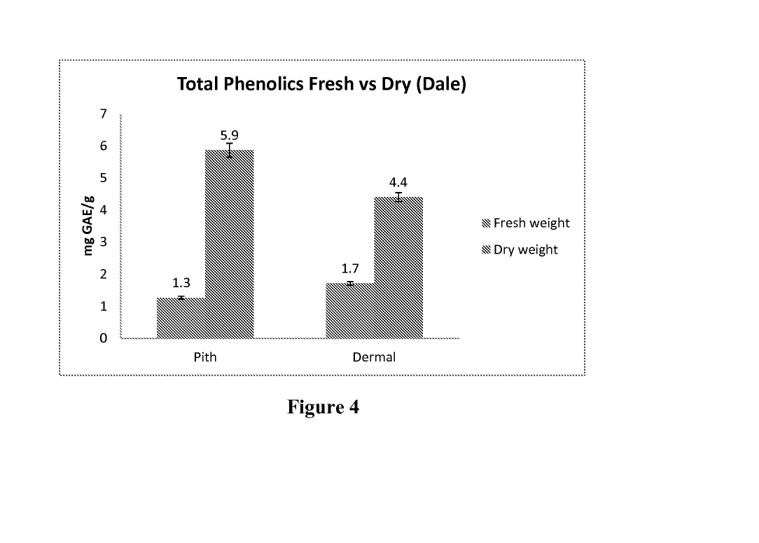

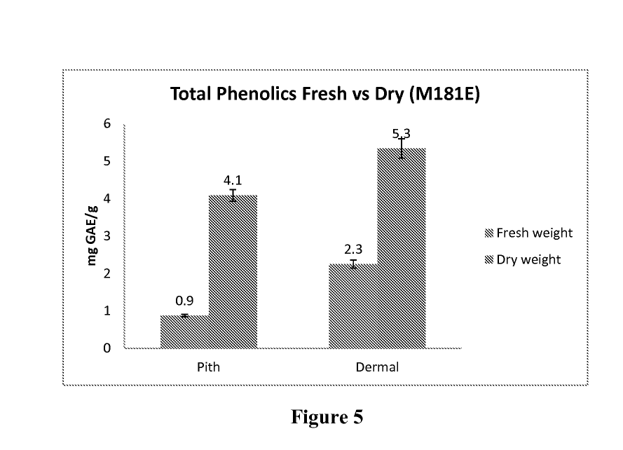

Methodology: Drying method used was lyophilization (e.g. freeze-drying). Weights were recorded before and after and percent moisture was calculated. The pith contained more water than dermal layer. See FIGS. 4, 5, and 6, and Table 3 for results.

Table 3: Displays results for the moisture analysis for pith and dermal layer of Dale and M81E variety. Results are presented as percent weight of moisture (% moisture)±standard error (SE).

TABLE 3

| Sample | % Moisture ± SE |
|---|---|
| Pith (Dale) | 78.4 ± 0.15 |
| Dermal (Dale) | 61.0 ± 0.66 |
| Pith (M81E) | 78.5 ± 0.28 |
| Dermal (M81E) | 57.8 ± 0.84 |

### Example 3

Assess difference in the metabolite profiles of sweet Sorghum extracts by LC-MSMS analysis. Identify phenolic compounds in sweet Sorghum extracts.

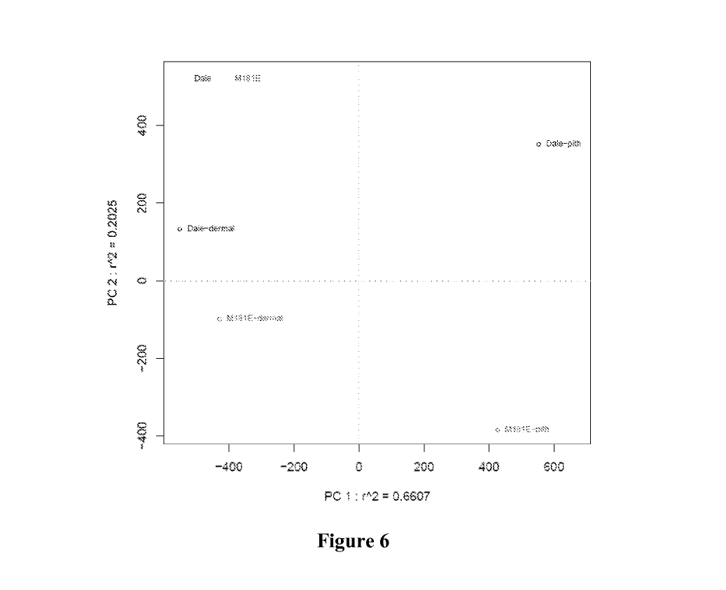

Methodology: Aliquots of extract were prepared and injected into a Water's Acquity UPLC coupled to a Water's Xevo G2 Q-Tof mass spec. Results shown in FIG. 6 and Table 4.

Table 4: Putative Sweet Sorghum Compounds Identified by LCMS Analysis

TABLE 4

| | | Rt | observed | exact | Δp | intensity^{c} | intensity^{c} | |
|---|---|---|---|---|---|---|---|---|
| identity | formula^{a} | (min) | mass | mass^{b} | pm | Dermal | Pith | ref^{d} |
| vanillic acid | C₈H₈O₄ | 2.79 | 169.050 | 169.0495 | 2 | 100.9 ± 3.8 | 68.2 ± 7.4* | ¹ |
| p-coumaric acid | C₉H₈O₃ | 0.85 | 165.055 | 165.0546 | 2 | 534.9 ± 14.6 | 492.0 ± 23.8 | ¹ |
| ferulic acid | C₁₀H₁₀O₄ | 3.12 | 195.067 | 195.0652 | 9 | 27.6 ± 1.8 | 14.3 ± 1.5* | ¹ |
| caffeic acid | C₉H₈O₄ | 3.16 | 181.050 | 181.0495 | 2 | 152.4 ± 1.3 | 26.5 ± 4.5* | ¹ |
| apigeninidin | C₁₅H₁₁O₄⁺ | 4.29 | 255.066 | 255.0660^{M} | 0 | 96.5 ± 20.2 | 7.9 ± 0.7* | ¹⁻³ |
| luteolinidin | C₁₅H₁₁O₅⁺ | 5.97 | 271.061 | 271.0607^{M} | 2 | 151.4 ± 30.8 | 7.8 ± 2.0* | ¹⁻³ |
| malvidin 3-O-glucoside | C₂₃H₂₅O₁₂⁺ | 4.76 | 493.134 | 493.1314^{M} | 3 | 168.6 ± 5.7 | 12.7 ± 1.4* | ³ |
| apigenin | C₁₅H₁₀O₅ | 4.04 | 271.061 | 271.0607 | 3 | 71.2 ± 53.8 | 8.6 ± 1.2 | ¹ |
| luteolin | C₁₅H₁₀O₆ | 5.48 | 287.056 | 287.0556 | 3 | 47.3 ± 54.2 | 4.6 ± 3.0 | ^{1,4} |
| trans-resveratrol | C₁₄H₁₂O₃ | 8.48 | 229.087 | 229.0859 | 4 | 28.8 ± 3.3 | 37.6 ± 2.9 | ¹ |
| luteoferol | C₁₅H₁₄O₆ | 4.26 | 291.087 | 291.0863 | 2 | 16.3 ± 0.8 | 6.4 ± 0.3* | |
| kaempferol | C₁₅H₁₀O₆ | 5.48 | 287.056 | 287.0556 | 3 | 47.3 ± 54.2 | 4.6 ± 3.0 | |

^{a}Formulas are based on [M].
^{b}Exact masses are based on [M + H]⁺ unless otherwise indicated.
^{M}Mass based on [M].
^{c}Normalized peak intensities based on peak areas normalized to total ion signal in R.
^{d}References which have previously identified the compound listed.
*Significant differences (P < 0.05) observed between dermal and pith extracts.

### Example 4

In vitro investigations of dermal extracts to determine an effective concentration range of Sorghum dermal extract that will induce apoptosis and suppress proliferation.

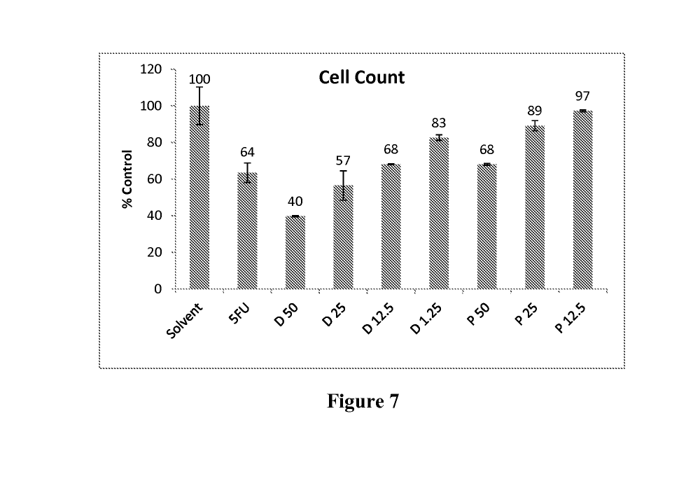

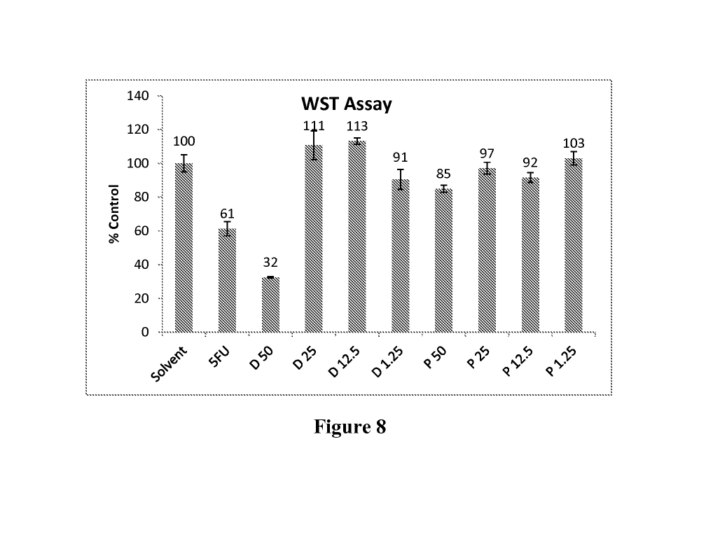

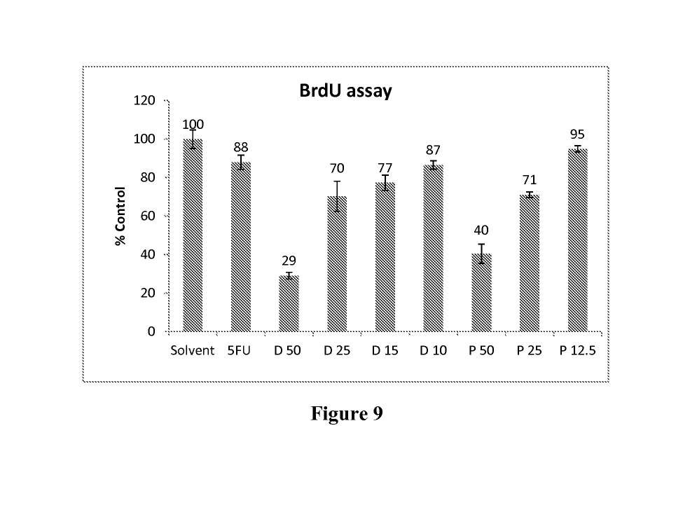

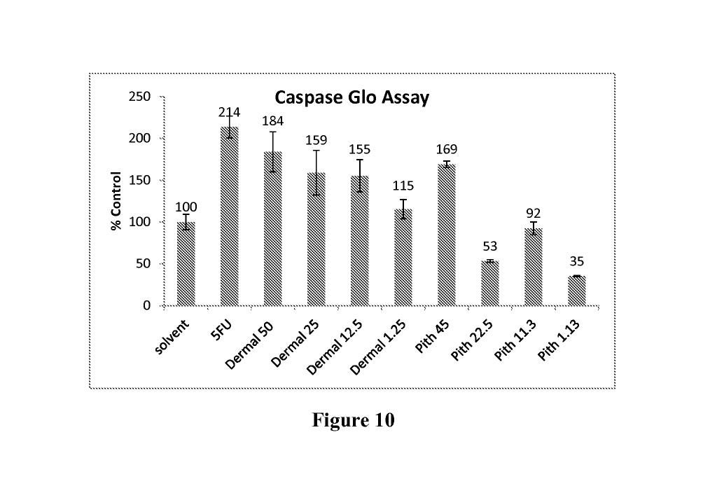

Methodology: Phenolic rich extracts were prepared by drying acetone extract and re-suspending it in a smaller volume of ethanol (used for its safety in cell culture assays when compared to acetone). The HCT 116 cell line was used as a model for early stage colon cancer. Cells were grown and treated with dermal and pith extracts from Dale variety for 24 hours before being assayed for cell proliferation and apoptosis. Cell proliferation was determined by two methods: i) Cell counting by cellometer; ii) viability by WST assay and iii) DNA synthetic rate by BrdU incorporation. Apoptosis was assessed by active caspase activity as measured by the Caspase Glo 3/7 assay. See results in FIGS. 7, 8, 9 and 10.

The results indicate that sweet Sorghum extracts have anti-cancer effects on HCT 116 cells in an in vitro model for early stage colon cancer. Pith and dermal extracts suppressed proliferation and induced apoptosis at concentrations ranging from 50 μg GAE/ml. Treatment with dermal extracts had the most effect in terms of suppressing proliferation and increasing apoptosis when compared to pith extracts. When compared to 5-fluorouracil (5FU) control, a standard anticancer drug, dermal extract was almost as effective in inducing apoptosis and even more effective in suppressing cellular proliferation.

### Example 5

Extraction Solvent

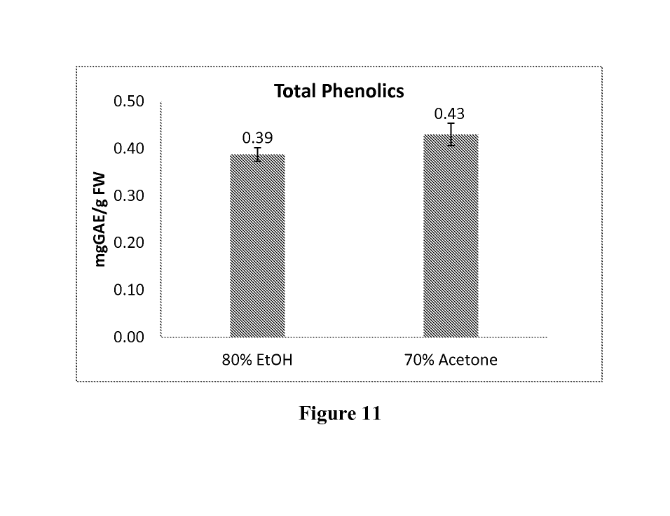

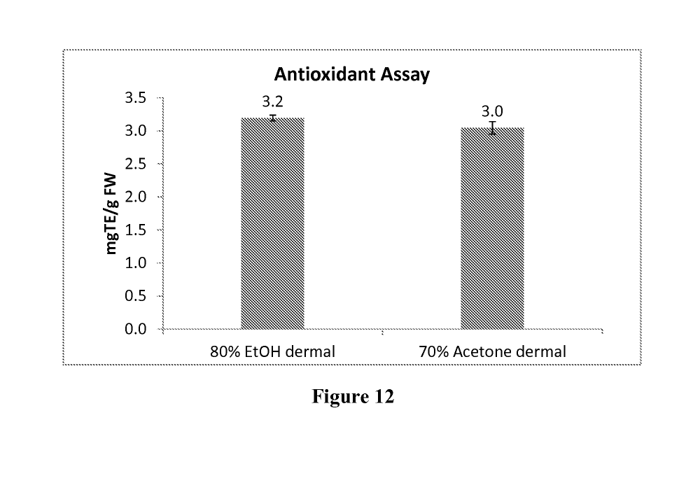

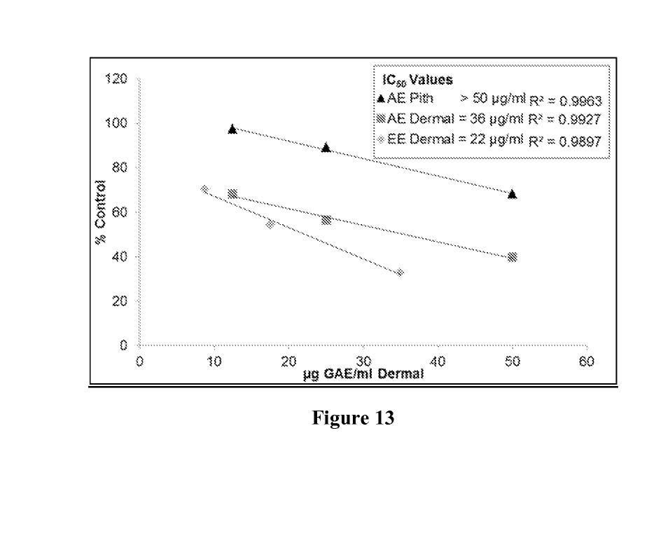

Liquid-liquid extraction was carried out using ethanol, 80% ethanol and 70% acetone as extraction solvents. Total phenolics and antioxidant activity was measured by the Folin Ciocalteu and ABTS assays, respectively. Anticancer activity was assessed by cell count comparing acetone extracts to ethanol extracts. See results in FIGS. 11, 12 and 13.

Pure ethanol makes a poor extraction solvent and yielded trace amounts of phenolics (data not shown). However, when water is added to ethanol at 20%, phenolic yield was increased to that of extraction with 70% acetone. This demonstrates that aqueous ethanol is an effective solvent to extract phenolic and antioxidant compounds from Sorghum . The extracts prepared with aqueous ethanol were more potent in suppressing the growth of cancerous HCT116 cells in vitro as determined by cell count.

### Example 6

Investigation to confirm anticancer properties in cancer stem cells by measuring proliferation employing cell count and BrdU assay, and by measuring apoptosis by caspase activity and TUNEL assay. Also determine if the extracts are acting through p53 dependent mechanisms by measuring apoptosis and proliferation in CSC p53 shRNA cells.

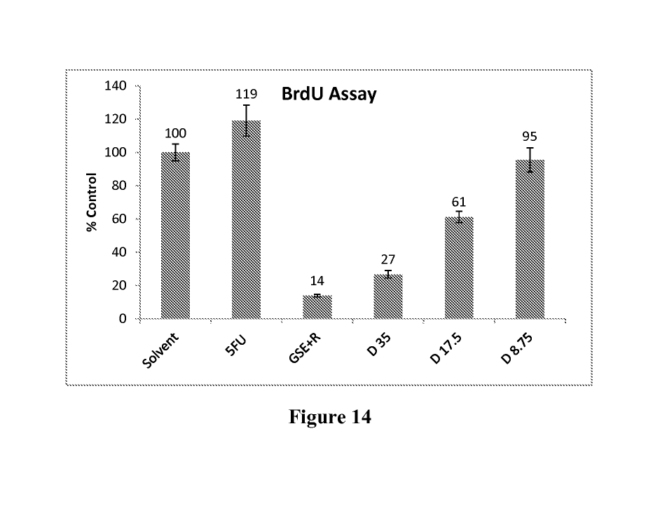

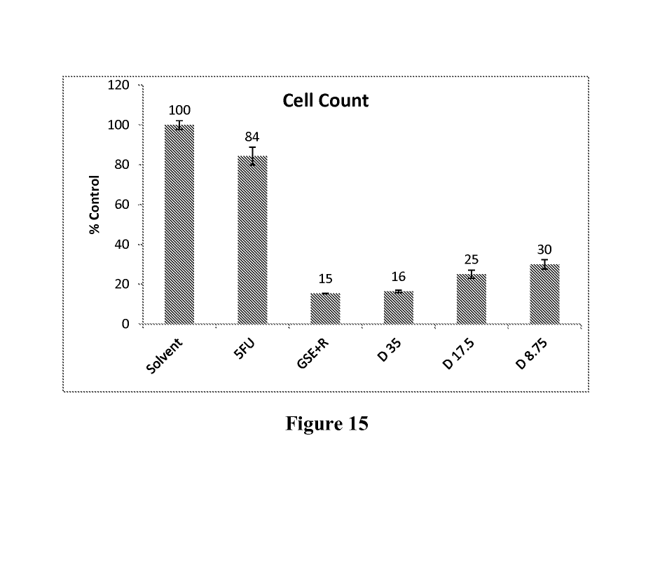

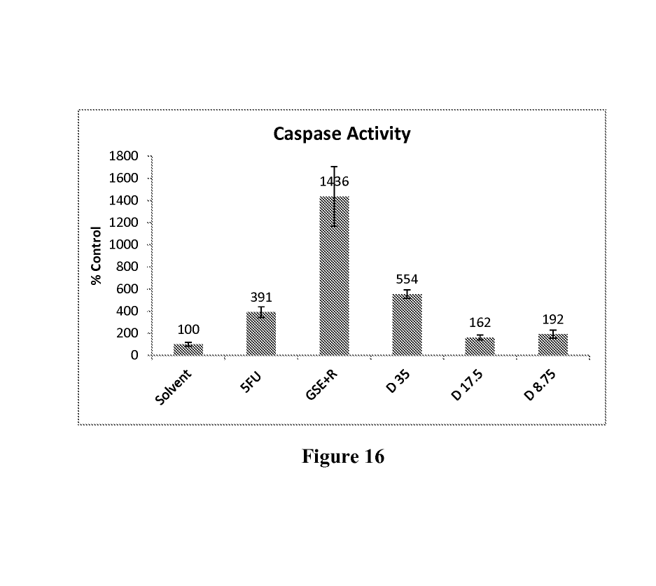

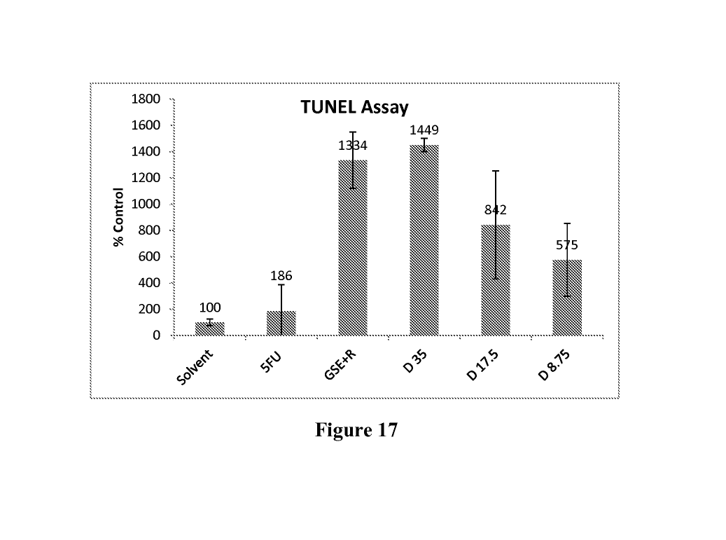

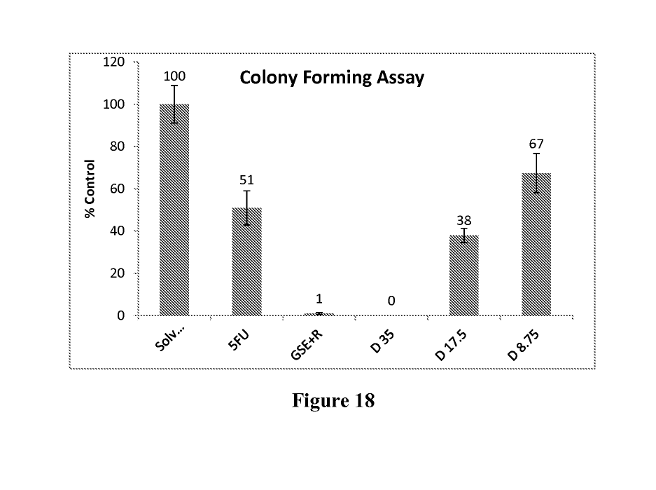

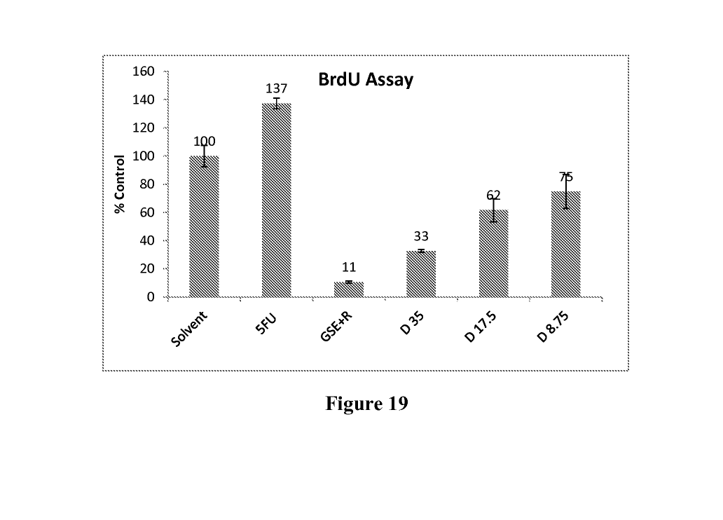

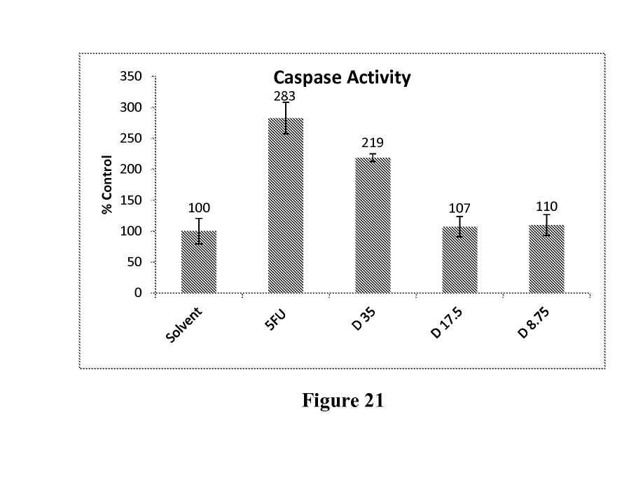

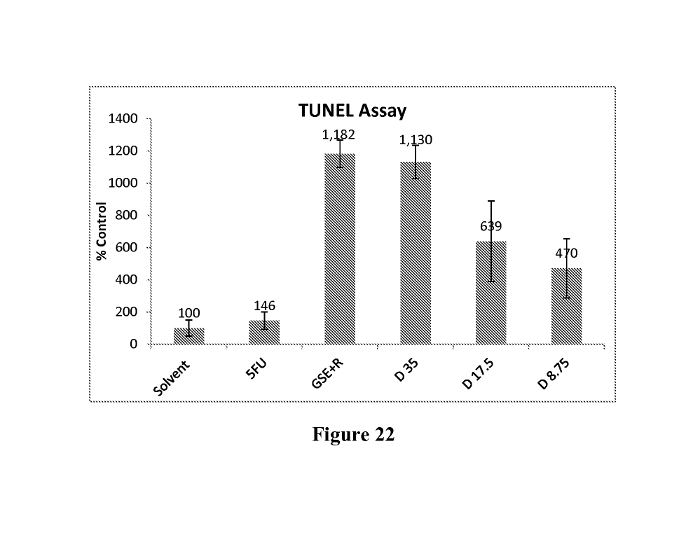

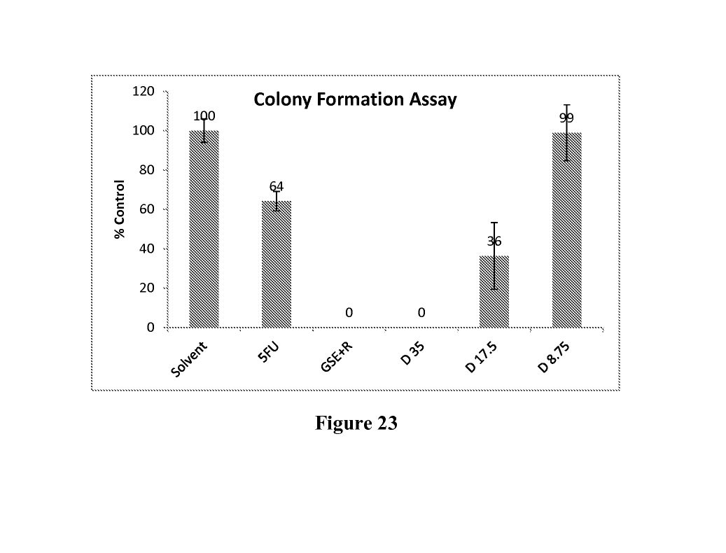

Methodology: CCSC and CCSC p53 shRNA cells will be treated with Sorghum Dale dermal extracts for 24 hours and assessed for proliferation by BrdU and cell count assays. Apoptosis will be measured by active caspase activity by the Caspase 3/7 Glo assay and apoptosis was confirmed with TUNEL assay. The stemness of the CCSC was also assessed by the Colony Formation Assay. All data are presented as % of the solvent control. See results in FIGS. 14-23.

Sweet Sorghum extracts showed anticancer properties to colon cancer stem cells in vitro by suppressing proliferation, increasing apoptosis and altering cell stemness. Sorghum was successful at targeting this resistant population. In many instances it demonstrated superior activity when compared to SFU, a standard chemotherapeutic drug. Furthermore, these anticancer mechanisms seem to be independent of p53 as shown by stem cells with suppressed p53 levels by shRNA. p53 is considered a tumor suppressor gene because it mediates cell cycle arrest and cell death. Loss of function leads to uncontrolled growth and suppressed cell death. In colorectal cancer, it is considered the second key step in tumorigenesis. Since it is a pivotal mutation in colorectal carcinogenesis, it is important to understand how cells respond to anti-cancer treatments in the absence of functional p53 activity. The anticancer activity may be due to the complex mixture of phenolic compounds which can act on multiple pathways in the cancer cell to induce cell death and arrest cell proliferation. This is in sharp contrast to conventional therapies that utilize single compounds in high concentrations, which indiscriminately targets cancer and normal cells. Ultimately, this results in the negative side effects of conventional chemotherapies.

### Example 7

Component Focused Analysis of Sweet Sorghum: Quantify total phenolics, antioxidants activity, and anticancer activity in the pith, dermal, seed head, leaf and whole plant of Sorghum grown in Fort Collins, Colo., and determine if single constituent or whole plant is superior in terms of anticancer activity.

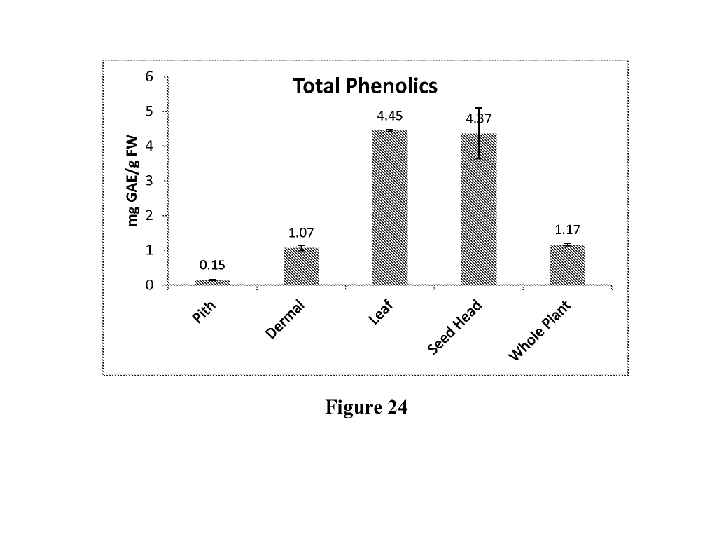

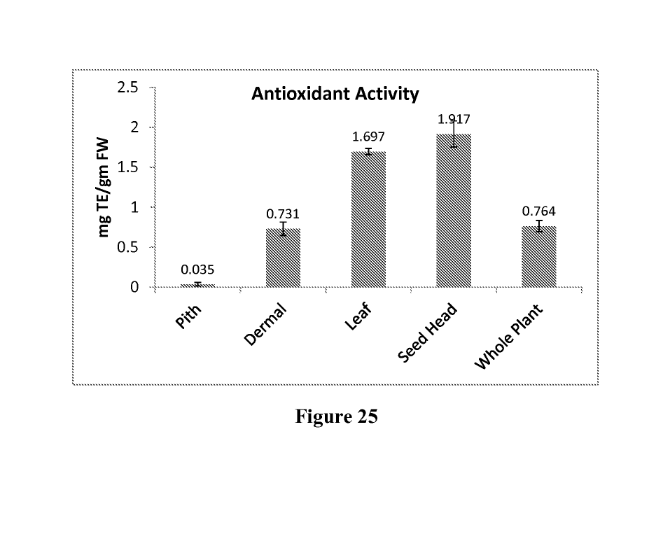

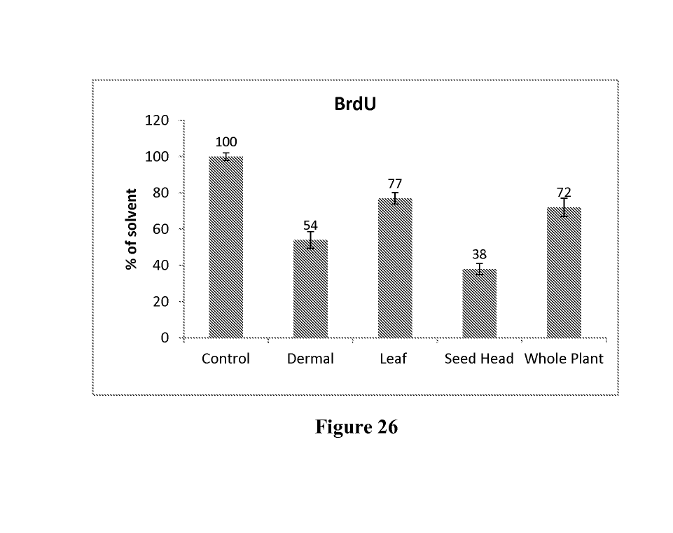

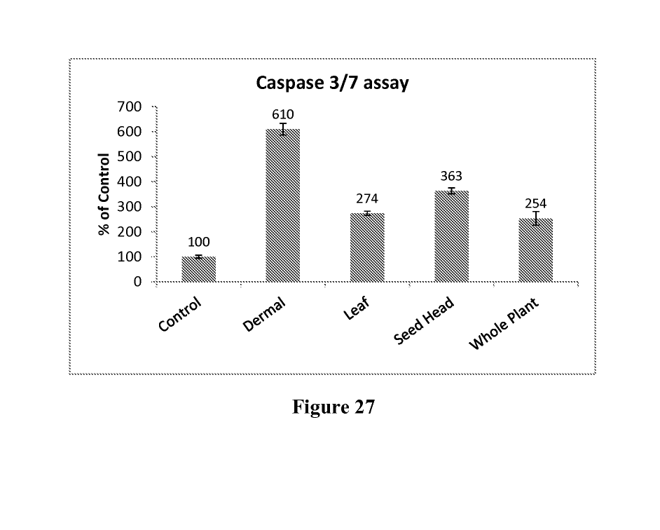

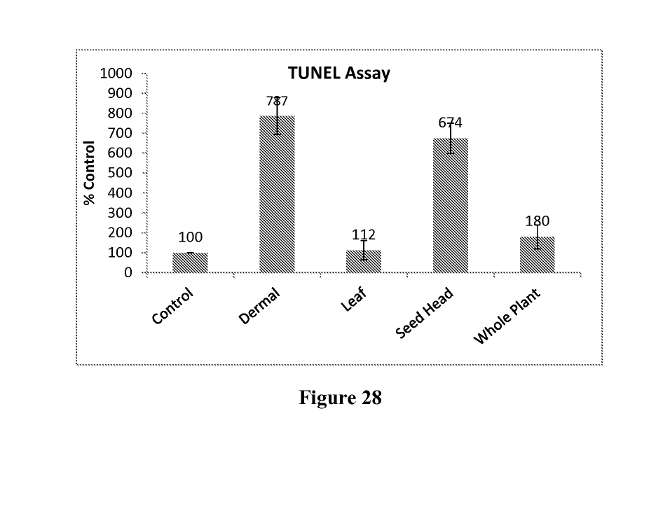

Methodology: Sorghum bicolor was grown and harvested in the Fall of 2012 (Fort Collins, Colo.) and processed into pith, dermal layer, leaf, and seed head. For whole plant, an entire plant was chopped into approximately one inch sections. The material was then frozen at −20° C. until analyzed for total phenolics, antioxidant activity and anticancer activity. To determine the phenolic and antioxidant activity in Sorghum components, extracts were prepared with 80% ethanol extraction solvent and total phenolics and antioxidants estimated with Folin-Ciocalteu and ABTS assays, respectively. Phenolic rich extracts of each component were then prepared and tested for in vitro anticancer activity by measuring cell growth (proliferation) and cell death (apoptosis). Cell growth was measured by BrdU assay. Apoptosis was measured by caspase 3/7 Glo and TUNEL assays. All extracts were dosed at 35 μg gallic acid equivalents per ml of media and cell growth and death were measured after 24 hours. See results in FIGS. 24-28.

Results from total phenolics and antioxidant activity analysis of the different Sorghum components revealed that all components of sweet Sorghum tested had measurable phenolics and antioxidant activity. The activity of the Sorghum components in decreasing order was seed head, leaf, dermal and pith. Whole plant is was a resultant of all 4 components analyzed and reflects the phenolic/antioxidant yield of a whole plant extraction.

Phenolic rich extracts prepared from sweet Sorghum components dermal and seed head exhibited superior activity to extracts prepared from leaf or from whole plant against colon cancer stem cells after 24 hours at same treatment level.

### Example 8

Seasonal and Varietal Analysis of Sweet Sorghum Seed Head and Dermal Layer: Determine the varietal differences between the dermal and seed head of different sweet Sorghum varieties, determine harvest-harvest variation in sweet Sorghum dermal and seed head harvested from two plantings at 4 stages of maturation and determine anticancer activities of these sweet Sorghum extracts in vitro against colon cancer stem cells.

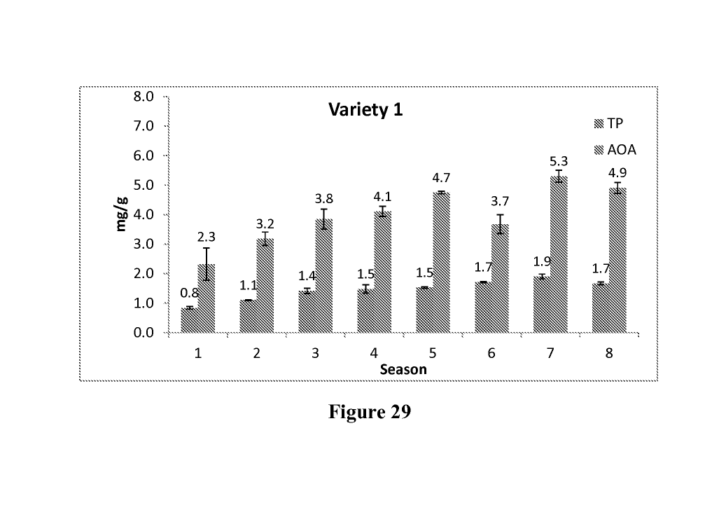

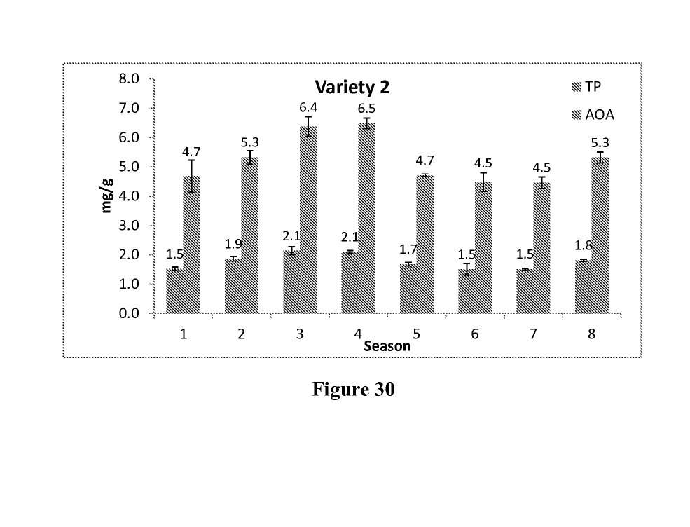

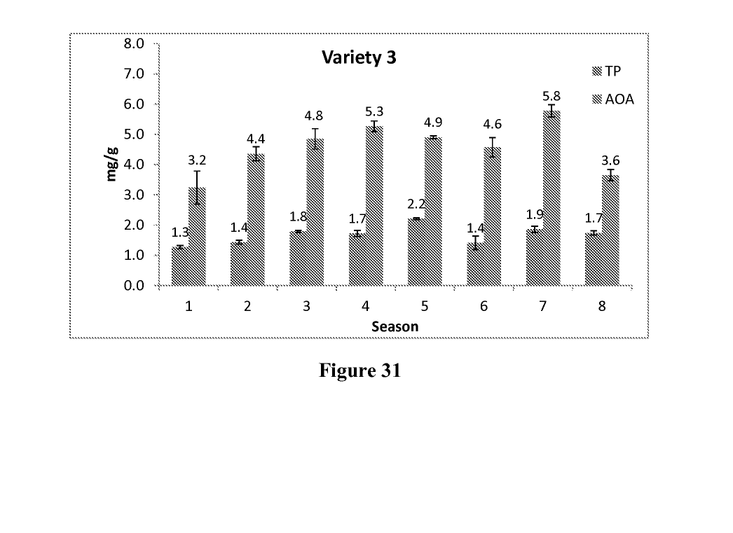

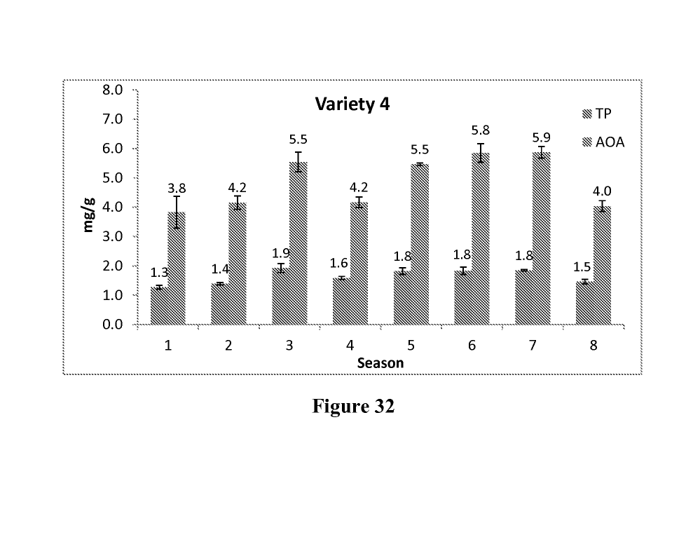

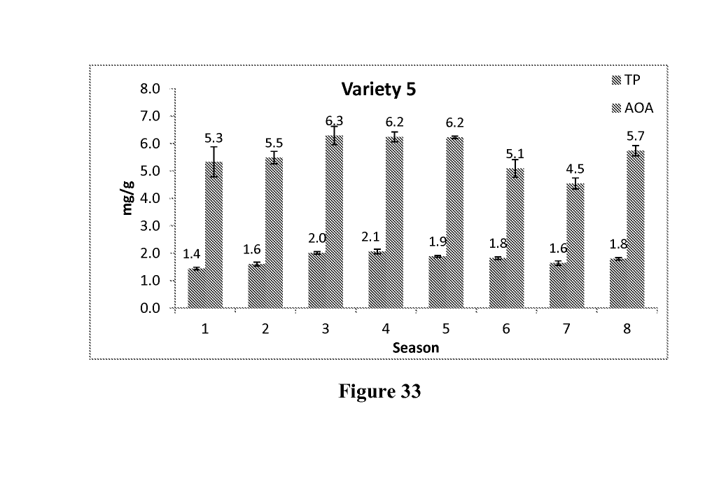

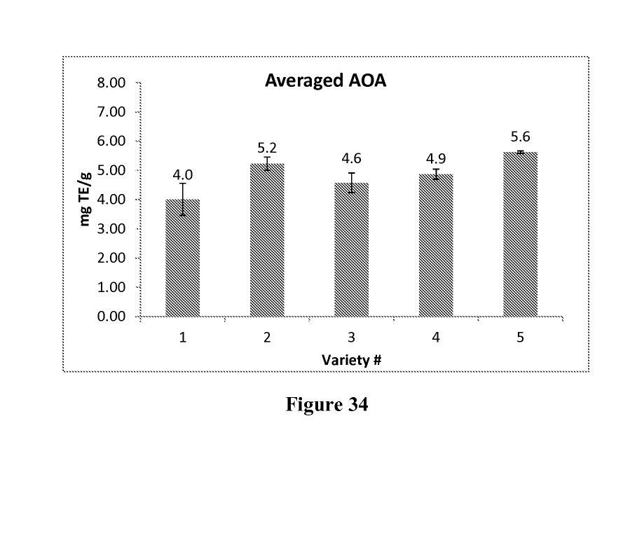

Methodology: Total phenolics and antioxidant activities were assessed as previously described. See results in FIGS. 29-35.

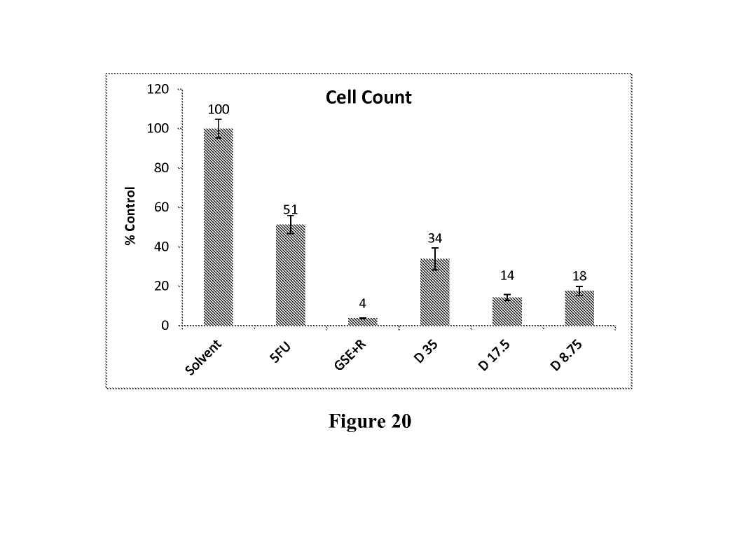

There was a linear increase observed in antioxidants and total phenolics from harvests 1-4 (indicated by arrows). On average across all 8 harvests, variety 5 had the highest antioxidant activity of the varieties tested (FIG. 20). This difference was significant for varieties 1 and 3 (P<0.05) and nearly significant to varieties 3 (P=0.08) and 4 (P=0.11).

The following publications, referred to in the description above by reference or number as set forth below, are hereby specifically incorporated by reference in their entirety:

1. Bröhan, M., Jerkovic, V. & Collin, S. Potentiality of red Sorghum for producing stilbenoid-enriched beers with high antioxidant activity. Journal of agricultural and food chemistry 59, 4088-94 (2011).

2. Njongmeta, N. Extractability profiling and antioxidant activity of flavonoids in Sorghum grain and non-grain materials. East (2010) at <http://gradworks.umi.com/33/70/3370773.html>.

3. Polycarpe Kayodé, A. P. et al. Uncommonly high levels of 3-deoxyanthocyanidins and antioxidant capacity in the leaf sheaths of dye Sorghum. Journal of agricultural and food chemistry 59, 1178-84 (2011).

4. Rey, J., Pousset, J., Levesque, J. & Wanty. Isolation and composition of a natural dye from the stem of Sorghum-Bicolor (L) Moench Subsp Americanum-Caudatum. Cereal Chemistry 70, 759-760 (1993).

What is claimed is:

1. A Sorghum bicolor (L.) Moench (Sorghum) extract, which satisfies the conditions (a) and (b):
a. comprising total phenolics of value, as determined by the Folin-Ciocalteu assay, at least greater than about 0.1 mg Gallic Acid Equivalent per gram Fresh Weight (GAE/g FW), and
b. antioxidant activity, as determined by the Trolox Equivalent Antioxidant Capacity (TEAC) method using ABTS (2,2′-azino-bis(3-ethylbenzthiazoline-6-sulfonic acid) as an colorimetric indicator compound, of at least greater than about 0.01 mg Trolox Equivalent per gram fresh weight (TE/g FW).

2. The Sorghum extract according to claim 1, wherein the total phenolics value is at least greater than about 4.0 mg GAE/g FW and the antioxidant activity is greater than about 0.4 mg TE/g FW.

3. The Sorghum extract according to claim 1, wherein the total phenolics value is at least greater than about 3.5 mg GAE/g FW and the antioxidant activity is greater than about 0.4 mg TE/g FW.

4. The Sorghum extract according to claim 1, wherein the total phenolics value is at least greater than about 0.9 mg GAE/g FW and the antioxidant activity is greater than about 0.5 mg TE/g FW.

5. A process for making a Sorghum extract comprising the steps of:
a. contacting a member selected from the group consisting of Sorghum whole plant, Sorghum grain, Sorghum seed head, Sorghum leaves, Sorghum pith, Sorghum juice, Sorghum dermal layer, Sorghum rind (with or without dermal layer removed) and mixtures thereof with a solvent at a temperature below 180 Fahrenheit such that certain compounds which are measurable under the Folin-Ciocalteu or TEAC assay methods are mass transferred from the member to the solvent; and
b. separating insoluble solids from the solvent obtained in step (a).

6. The process according to claim 5, wherein the solvent is selected from the group consisting of alcohol, supercritical CO₂, acetone, water and hexane or a mixture thereof.

7. The process according to claim 5, wherein the solvent is aqueous ethanol.

8. The process according to claim 7, wherein the solvent is about 80% aqueous ethanol.

9. The process according to claim 5, further comprising the steps of grinding of the said members before or after contacting said members with the solvent.

10. The process according to claim 5, further comprising the steps:
a. selectively removing the solvent through methods such as settling, filtration, evaporation and the like; and
b. obtaining the Sorghum extract.

11. The process according to claim 5, further comprising the steps:
c. further purifying the product of step (a) through methods such as chromatography, adsorption, and the like; and
d. obtaining the Sorghum extract.

12. The process according to claim 5, further comprising the step of purifying the crude Sorghum extract to obtain a purified Sorghum extract.

13. The process according to claim 5, wherein the Sorghum extract comprises total phenolics further wherein the total phenolics as determined by the Folin-Ciocalteu assay have a value at least greater than about 0.1 mg GAE/g FW.

14. The process according to claim 5, wherein the Sorghum extract exhibits an antioxidant activity as determined by the ABTS assay of at least greater than about 0.01 mg TE/g FW.

15. The process according to claim 5, wherein the member is Sorghum leaf.

16. The process according to claim 15, wherein the Sorghum leaf comprises total phenolics further wherein the total phenolics as determined by the Folin-Ciocalteu assay have a value at least greater than about 4.0 mg GAE/g FW.

17. The process according to claim 15, wherein the Sorghum leaf exhibit an antioxidant activity as determined by the ABTS assay of at least greater than about 0.4 mg TE/g FW.

18. A composition comprising the Sorghum extract according to claim 1.

19. A composition of claim 18, wherein the composition comprises one or more of a beverage, nutraceutical, injection, or inhalation.

20. A composition according to claim 1, further comprising an excipient, diluent or carrier.
